(19) United States
(12) Patent Application Publication
(10) Pub. No.: US 2026/0102261 A1
(43) Pub. Date: Apr. 16, 2026

(54) PROSTHETIC FOOT WITH COMPOSITE ATTACHMENT

(71) Applicant: WillowWood Global LLC, Mount Sterling, OH (US)

(72) Inventors: Monty Allen Moshier, Queen Creek, AZ (US); Evandro M. Ficanha, Grove City, OH (US); Matthew M. Wernke, Tampa, FL (US); James M. Colvin, Hilliard, OH (US); Jayanth Bangalore Manjunath, Mesa, AZ (US)

(21) Appl. No.: 19/411,344

(22) Filed: Dec. 7, 2025

Related U.S. Application Data
(63) Continuation-in-part of application No. 17/509,864, filed on Oct. 25, 2021.
Application No. 17/509,864 is a continuation-in-part of application No. 16/808,796, filed on Mar. 4, 2020, now Pat. No. 11,173,053.

(51) Int. Cl. A61F 2/50; A61F 2/66

(52) U.S. Cl. CPC A61F 2/5044; A61F 2/66; A61F 2002/5055; A61F 2002/6678

(57) ABSTRACT

A composite prosthetic foot assembly comprising a composite foot plate, a composite shank plate, and a composite attachment structure. The composite attachment structure attaches the shank plate to the foot plate and includes a portion configured to extend transversely through at least the foot plate and the shank plate. The composite attachment structure is made of a plurality of elongated fibers surrounded by a resin material, and has a first end portion and a second end portion, with at least a portion of the plurality of elongated fibers configured to extend continuously through the shank plate and the foot plate, with the first end disposed within a first recess formed in the foot plate and the second end disposed within a second recess formed in the shank plate. A composite prosthetic foot assembly comprising a heel plate, a shank plate, and composite attachment structure attaching the two is also disclosed.

### RELATED APPLICATIONS

This application is a continuation-in-part of U.S. patent application Ser. No. 17/509,864, filed Oct. 25, 2021, which is a continuation-in-part of U.S. patent application Ser. No. 16/808,796, filed on Mar. 4, 2020 and issued as U.S. Pat. No. 11,173,053.

### STATEMENT OF FEDERALLY FUNDED RESEARCH

This invention was made with government support under 2R42HD093476-02A1 awarded by the National Institutes of Health, Eunice Kennedy Shriver National Institute of Child Health & Human Development. The government has certain rights in the invention.

### TECHNICAL FIELD

The disclosure relates to prosthetic limbs for humans, and more particularly, to a prosthetic foot.

### BACKGROUND

A conventional prosthetic foot design may have structural components including a foot plate with a toe portion, a heel portion, and an intermediate portion with an arched configuration that flexes under the weight of the user as needed throughout the gait cycle. The structural components also may include a shank plate in addition to the foot plate. The shank plate typically has a vertical upper portion for connection with another prosthetic device such as, for example, a prosthetic knee or a socket. The shank plate also may have a lower portion with a curvature reaching forward from the upper portion. The lower portion of the shank plate flexes under the weight of the user and is fastened to the foot plate to transmit the weight load forces to the foot plate.

Although such designs are lightweight and relatively inexpensive to fabricate, they may be subject to failure after repeated use when the connection between the shank plate and the foot plate degrades or separates. Also, such designs may be difficult to customize to suit the needs of a user. Accordingly, there is a need for a robust, easily customized prosthetic foot.

### SUMMARY

In embodiments, a composite prosthetic foot assembly includes a composite foot plate having a toe portion, a heel portion, an upper surface, a lower surface, a first aperture extending through the foot plate from the upper surface of the foot plate to the lower surface of the foot plate, and a first recess formed in the lower surface of the foot plate about the first aperture. The assembly further includes a composite shank plate having a toe portion, an upper portion, an upper surface, a lower surface, and a second aperture extending through the shank plate from the upper surface of the shank plate to the lower surface of the shank plate, and a second recess formed in the upper surface of the shank plate about the second aperture. In some embodiments, the shank plate is configured to be attached to the foot plate with a composite attachment structure along a seam that extends from the toe portion of the foot plate rearward toward the heel portion of the foot plate, the seam terminating at an end. The shank plate may be configured to be spaced apart from the foot plate at a gap between the foot plate and shank plate that extends rearward from the end of the seam.

In some embodiments, the composite attachment structure includes a portion configured to extend transversely through at least the foot plate and the shank plate. The composite attachment structure may have a first end portion and a second end portion, and comprises a plurality of elongated fibers within a resin material. At least a portion of the plurality of elongated fibers (together with the resin material in which such fibers are embedded or otherwise disposed) is configured to extend continuously through the shank plate and the foot plate, and may be disposed in a direction that is substantially perpendicular to the seam. In various embodiments, the elongated fibers may be made of a material selected from one or more of steel, carbon, glass, nylon, an aramid, and polyethylene.

In some embodiments, the composite attachment structure is configured to extend through at least the foot plate and the shank plate, wherein the first and second end portions of the composite attachment structure extend from the first and second apertures of the plates, respectively, and are folded (unidirectionally or multidirectionally) over the first and second recesses of the plates, respectively, to form enlarged end portions. The enlarged end portions may be molded, formed, or otherwise configured to at least partially or completely fill (or, potentially, overfill) the first and second recesses, or, optionally, the enlarged end portions may be capped by one or more separate layers of composite material comprising reinforcing fibers (including linear, substantially linear, and/or non-linear filament structures) and resin material that are molded, formed, or otherwise configured to at least partially or completely fill (or, potentially, overfill) the first and second recesses.

In some embodiments, the first recess may be rectangular in shape and extend across a majority of, substantially all of, or the entirety of a width of the foot plate. The second recess may also or individually be rectangular in shape and extend across a majority of, or substantially all of, or the entirety of a width of the shank plate. In other embodiments, one or both of the recesses and/or apertures may be circular or oval or polygonal in shape. Depending on the shape of the recesses and/or apertures, the recesses may be dimensioned such that one or more of their width, length, or diameter is multiple times larger than one or more of the width, length, or diameter of the apertures.

In some embodiments, the first recess and first aperture in the foot plate and the second recess and second aperture in the shank plate form an inner opening. In other embodiments, the first recess in the foot plate may have a first plurality of apertures and the second recess in the shank plate may have a second plurality of apertures aligned with the first plurality of apertures, and which recesses and apertures collectively form the inner opening. The inner opening may have any centrally narrowed or narrowing cross-sectional shape, including bilaterally stepped, tapered, helical, curved, and beveled cross-sectional shapes and combinations thereof, and the inner surfaces of the inner opening may be textured. The composite attachment structure may be molded, formed, or otherwise configured to at least partially or completely fill (or, potentially, overfill) the inner opening. Optionally, one or more of the recesses may be capped by one or more separate layers of composite material comprising reinforcing fibers (including linear, substantially linear, and/or non-linear filament structures) and resin material that are molded, formed, or otherwise configured to at least partially or completely fill (or, potentially, overfill) such recesses. In some embodiments, the composite attachment structure may be, at least in part, woven through the plurality of opposing apertures.

In some embodiments, the assembly further includes a second composite attachment structure disposed through at least the shank plate. In some embodiments, the second composite structure is also configured to extend through the foot plate. In other embodiments, the second composite attachment structure is configured to secure an adapter disposed at the upper portion of the shank plate. Optionally, the assembly may further comprise a composite backing plate disposed along the lower surface of the upper portion of the shank plate; and the second composite attachment structure is configured to extend through the backing plate, the shank plate, and the adapter. In some embodiments, the second composite attachment structure is a U-shaped structure that is configured to extend through the shank plate (and composite backing plate, if present) and through a central body of the adapter. In other embodiments, the second composite attachment structure is a U-shaped structure that is configured to extend through the shank plate (and composite backing plate, if present) and around at least a portion of a central body of the adapter.

Other objects and advantages of the disclosed composite prosthetic foot structure will be apparent from the following description, the accompanying drawings, and the appended claims.

### BRIEF DESCRIPTION OF THE DRAWINGS

FIG. 1 is a side view in section of an embodiment of the disclosed composite prosthetic foot structure;

FIG. 2 is an exploded view showing additional parts of the prosthetic foot structure of FIG. 1, shown between mold parts;

FIG. 3 is a detail view showing a composite attachment structure inserted through the shank plate and the foot plate of the composite prosthetic foot structure of FIG. 1 prior to molding;

FIG. 4 is an enlarged view of the composite attachment structure of the composite prosthetic foot structure of FIG. 1;

FIG. 5 is an enlarged view of an alternative attachment structure of the composite prosthetic foot structure of FIG. 1;

FIG. 6 is a schematic side elevation of an alternative embodiment of the disclosed composite prosthetic foot structure;

FIG. 7 is a schematic side elevation of a second alternative embodiment of the disclosed composite prosthetic foot structure;

FIG. 8 is a schematic side elevation of a third alternative embodiment of the disclosed composite the prosthetic foot structure;

FIG. 9 is an exploded view of an embodiment that includes the prosthetic foot structure 10D of FIG. 13, shown between mold parts;

FIG. 10 is a detail view showing the composite attachment structure of a fourth alternative embodiment of the disclosed prosthetic foot structure;

FIG. 11 is a detail view showing the composite attachment structure of a fifth alternative embodiment of the disclosed prosthetic foot structure;

FIG. 12 is a detail view showing the composite attachment structure of a sixth alternative embodiment of the disclosed prosthetic foot structure;

FIG. 13 is a detail view showing the composite attachment structure of a seventh alternative embodiment of the disclosed prosthetic foot structure;

FIG. 14 is a detail view showing the composite attachment structure of an eighth alternative embodiment of the disclosed prosthetic foot structure;

FIG. 15 is a detail view showing the composite attachment structure of a ninth alternative embodiment of the disclosed prosthetic foot structure;

FIGS. 16A-D are top views of an exemplary prosthetic foot structure showing various embodiments of a composite attachment structure, with FIG. 16 C being a partially exploded top view;

FIGS. 17A-E are cross-sectional detail views of various embodiments of an inner opening and a composite attachment structure;

FIG. 18A and B are cross-sectional detail views showing inner surfaces of an inner opening formed through the foot plate and the shank plate;

FIGS. 19A-C include a perspective, cross-sectional side, and bottom view, respectively, of an embodiment of the disclosed composite prosthetic foot structure;

FIGS. 20A-C includes a perspective, cross-sectional side, and bottom view, respectively, of an alternative embodiment of the disclosed composite prosthetic foot structure;

FIGS. 21A-D include a perspective, cross-sectional side, rear, and front view, respectively, of another alternative embodiment of the disclosed composite prosthetic foot structure;

FIG. 22A and B include a cross-sectional side view and a rear view, respectively, of yet another alternative embodiment of the disclosed composite prosthetic foot structure;

FIGS. 23A-E includes a perspective, rear, front, cross-sectional side, and transverse cross-sectional view, respectively, of a further alternative embodiment of the disclosed composite prosthetic foot structure that includes a U-shaped composite attachment structure;

FIGS. 24A-D includes a perspective, front, cross-sectional side, and transverse cross-sectional view, respectively, of a yet further alternative embodiment of the disclosed composite prosthetic foot structure that includes a U-shaped composite attachment structure; and

FIG. 25 includes a cross-sectional side view of an alternative embodiment of the disclosed composite attachment structure applied to the disclosed composite prosthetic foot structure of FIG. 6.

### DETAILED DESCRIPTION

The structures illustrated in the drawings include parts that are examples of the structural elements recited in the claims. One or more elements of one embodiment may be used in combination with, or as a substitute for, one or more elements of another as needed for any implementation of the claimed invention.

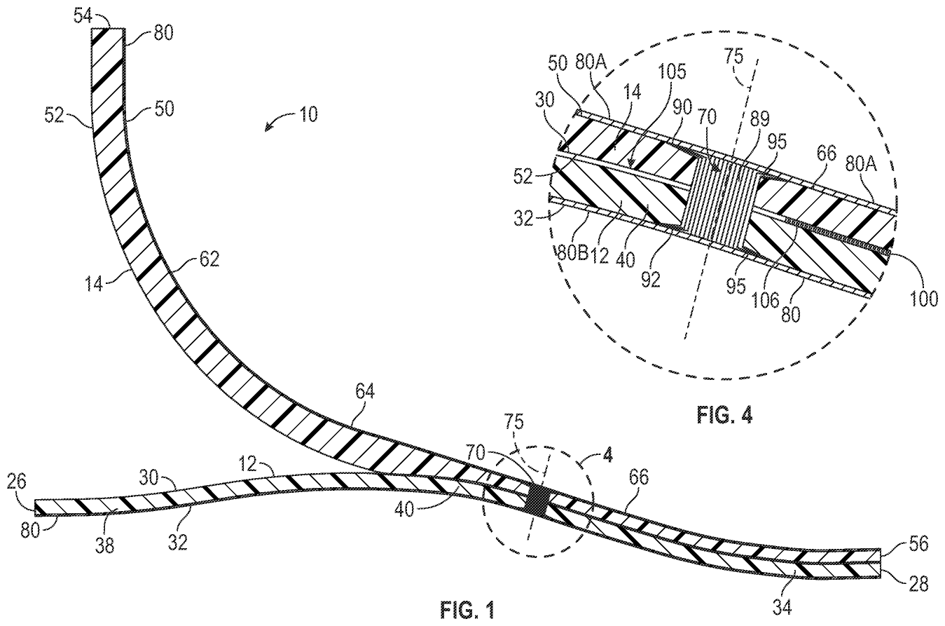

As shown in FIG. 1, an exemplary embodiment of the disclosed prosthetic foot, generally designated 10, includes a plate assembly comprising a foot or base plate 12 and a shank plate 14. The prosthetic foot 10 is adapted to be combined with other parts (not shown) of a complete prosthetic foot system. Such other parts may include, for example, a cosmetic foot shell. The foot plate 12 and the shank plate 14 both have strength and stiffness as needed to support the user's weight and have flexibility as needed to serve as springs for comfort and assistance in the heel-strike and toe-off stages of the user's gait cycle.

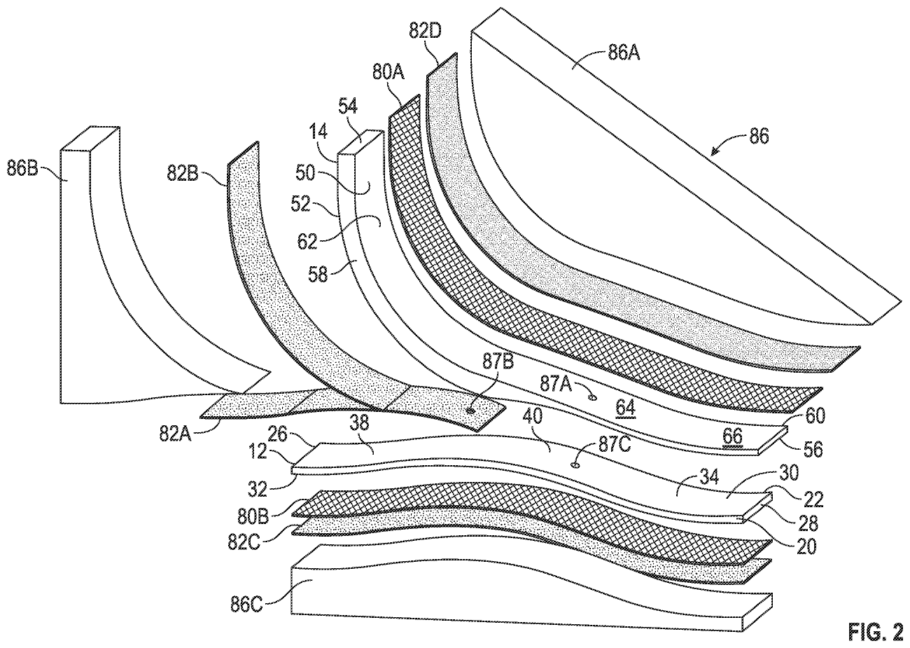

As shown in FIGS. 1 and 2, in embodiments, the foot plate 12 has a narrow, elongated shape with opposing side edges 20 and 22 reaching longitudinally between opposing end edges 26 and 28. Top and bottom side surfaces 30 and 32, respectively, define the length L of the foot plate 12 between the opposing end edges 26 and 28, as well as the width W of the foot plate 12 between the opposing side edges 20 and 22. In embodiments, the top and bottom side surfaces 30 and 32 have contours defining generally distinct length portions of the foot plate 12, including a toe portion 34, a heel portion 38, and an arched intermediate portion 40.

In embodiments, the shank plate 14 also has an elongated shape with top and bottom side surfaces 50 and 52, respectively, defining its length L between opposing end edges 54 and 56 and width W between opposing side edges 58 and 60. The top and bottom side surfaces 50 and 52 have contours defining an upper portion 62, an intermediate portion 64, and a lower portion 66 of the shank plate 14.

In one embodiment, both the foot plate 12 and the shank plate 14 are formed of a composite material including reinforcing fibers embedded in a resin material. In an exemplary embodiment, the fibers are aligned primarily unidirectionally lengthwise of each plate 12 and 14. However, it should be understood that the reinforcing fibers may be configured to be aligned at any suitable angle, relative to the length L of each plate 12 and 14. In one embodiment, the fibers of the foot plate 12 and the fibers of the shank plate 14 are aligned in substantially the same direction. In another embodiment, the fibers of each plate, 12 and 14, are aligned in substantially different directions, relative to the length L.

In one embodiment, the composite foot plate 12 and composite shank plate 14 are made of carbon fiber reinforced epoxy composite and cured in an autoclave or by other suitable processes. It should be appreciated that the foot plate 12 and the shank plate 14 may be fully cured prior to being attached to one another with a composite attachment structure, or one or both of the foot plate 12 and the shank plate 14 may be uncured and cured together with an uncured composite attachment structure during the manufacturing process. Other suitable methods for fabricating the plates 12 and 14 include compression and/or heat molding and resin transfer molding.

In embodiments, a composite attachment structure 70 is configured to attach the shank plate 14 to the foot plate 12 at various attachment points and configurations. In the configuration shown in FIG. 1, the upper portion 62 of the shank plate 14 is oriented vertically for connection with another component of a prosthetic leg, such as a socket or a knee joint (not shown). The lower portion 66 of the shank plate 14 is configured to overlay the intermediate and toe portions 40 and 34 of the foot plate 14. The curvature of the intermediate portion 64 of the shank plate 14 provides a flexible transition between the upper portion 62 and the lower portion 66. The composite attachment structure 70 may be configured to attach the lower portion 66 of the shank plate 14 to the intermediate portion 40 of the foot plate. However, it should be appreciated, as will be described in more detail below, that one or more composite attachment structures 70 may be disposed at various positions along the prosthetic foot 10 and in various configurations.

The composite attachment structure 70 generally comprises a composite material including reinforcing fibers (such as linear, substantially linear, or non-linear filament structures) within a resin material. In one embodiment, the composite attachment structure is made of a carbon fiber reinforced epoxy composite. Other fiber reinforcements with appropriate strength and flexibility properties, such as aramid (e.g. Kevlar, Nomex, Twaron), fiberglass, (e.g. E-glass, S-glass, C-glass), boron, nylon, polyethylene (including HDPE and UHMWPE, commercially available from Spectra and Dyneema), polypropylene (e.g. Innegra), PBO (e.g. Zylon), quartz, ceramic, graphene, basalt, metal (e.g. stainless steel and steel), and natural fibers (e.g. bamboo, flax, hemp, cotton, kenaf), could be used. Other resins with appropriate bonding, strength, and durability properties, such as urethane, polyethylene, polyester, vinyl ester, polypropylene, polyamide, PEEK, PLA, phenolic, cyanate esters, and acrylic, could be used.

The fibers in the composite attachment structure 70 may be primarily aligned unidirectionally along a transverse axis 75 normal to the lengthwise directions of the foot and shank plates 12 and 14. The fibers in the composite attachment structure 70 may thus be configured to reach through apertures 87 in the plates 12 and 14 primarily in a common direction through the thickness of the plates 12 and 14.

As shown in FIG. 2, in embodiments, the foot structure 10 includes additional parts, such as layers 80 of composite weave material. One layer 80A of composite weave material is provided to overlie the top surface 50 of shank plate 14. Another layer of composite weave material 80B is provided to underlie the bottom surface 32 of the foot plate 12. In embodiments, additional parts also include layers of peel ply film 82A, 82B, 82C, 82D that are placed between the plates 12 and 14 and surrounding parts of mold 86, which in embodiments include upper part 86A, heel part 86B, and sole part 86C.

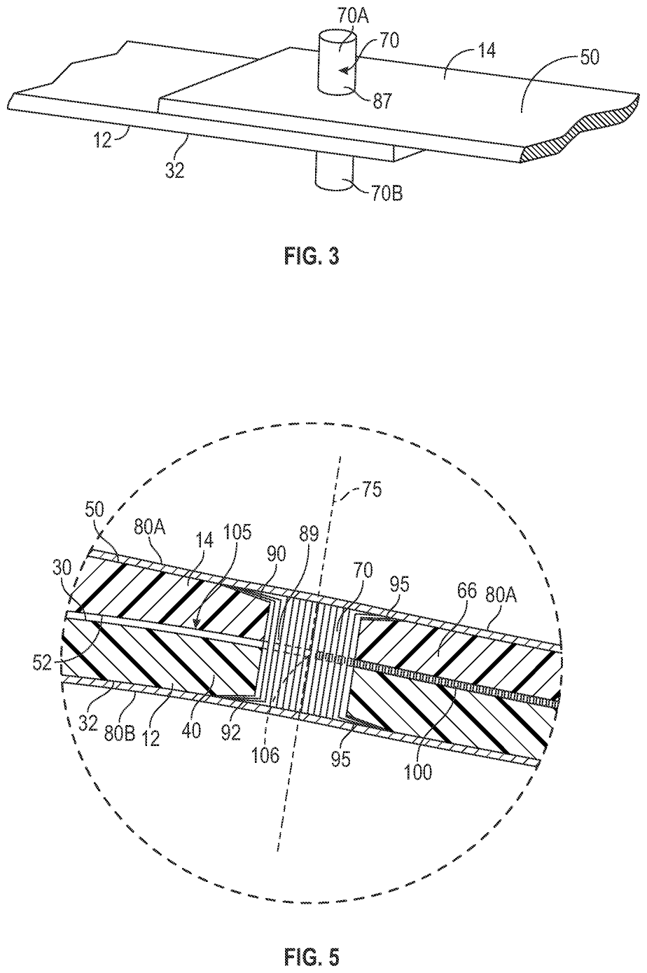

As shown in FIG. 2, in embodiments, the foot plate 12, the shank plate 14, and, optionally, an intervening layer 82A of peel ply film, are provided with apertures 87. As shown in FIG. 3, in embodiments the composite attachment structure 70 takes the form of a sheet of composite material, such as the composite material used to form the foot plate 12 and/or the shank plate 14, that is rolled into the shape of a cylinder. In one example, when the components of FIG. 2 are placed together in the mold, the apertures 87 are aligned on the transverse axis 75 (FIG. 4), and the cylindrical composite attachment structure 70 is inserted though the aligned apertures 87. In embodiments, the cylindrical composite attachment structure 70 is shaped such that upper and lower portions 70A, 70B, respectively, protrude above the top surface 50 of the shank plate 14 and below the bottom surface 32 of the foot plate 12 and form the top and bottom flattened end portions 90, 92, respectively, described below with reference to FIG. 4, interconnected by a cylindrical central shaft 89.

In an embodiment depicted in FIG. 2, the peel ply film 82D, composite weave material layer 80A, shank plate 14, peel ply films 82B, 82A, foot plate 12, composite weave material layer 80B, and peel ply film 82C are layered as shown and the mold parts 86A, 86B, and 86C are brought together and the composition heated. This causes the composite weave material layers 80A, 80B to bond to the shank plate 14 and foot plate 12, respectively, the shank plate 14 to bond to the foot plate 12 along a seam 100 (FIG. 4), and the shank plate 14 and foot plate 12 to conform to the contours of the mold parts 86A, 86B, and 86C, resulting in the shape of the composite prosthetic foot structure 10 shown in FIG. 1.

In an embodiment, this molding process also deforms the composite attachment structure 70 into the configuration shown in FIG. 4, in which the top and bottom portions 70A, 70B are compressed and flatten into flattened or flared top and bottom end portions 90 and 92, respectively. As indicated schematically in FIG. 4, in an exemplary embodiment, the reinforcing fibers in the top and bottom end portions 90 and 92 may be folded (unidirectionally or multidirectionally (e.g., flared or splayed radially)) or may otherwise diverge radially outward, over the top surface 50 of the shank plate 14 and the bottom surface 32 of the foot plate 12. In an embodiment, the top and bottom surfaces 50 and 32 of the plates 14 and 12, respectively, are provided with recesses, or channels, 95 that receive the folded over end portions 90 and 92 of the composite attachment structure 70, as shown in FIG. 4, which may avoid the formation of a surface bulge from the top surface 50 and bottom surface 32 of the shank plate 14 and foot plate 12 at each of those locations. The recesses 95 may be any size and shape suitable to at least accommodate the fibers and resin of the deflected end portions 90 and 92 of the composite attachment structure 70. The reinforcing fibers in the top and bottom end portions 90 and 92 may accordingly be folded (unidirectionally or multidirectionally (e.g., flared or splayed radially)) over the recesses 95 of the top surface 50 of the shank plate 14 and the bottom surface 32 of the foot plate 12, respectively.

In yet another embodiment, the foot plate 12 and the shank plate 14 are preformed and cured before the composite attachment structure 70 is inserted. In this embodiment, the uncured composite material of the composite attachment structure 70 is compression and/or heat molded after placement for curing of the composite attachment structure 70 and permanent attachment of the foot plate 12 to the shank plate 14. Whether the plates 12 and 14 and the composite attachment structure 70 are cured together or separately, as the composite attachment structure 70 is cured, the top and bottom portions 70A, 70B are compressed and flatten into flattened, flared, or enlarged top and bottom end portions 90 and 92, as described above.

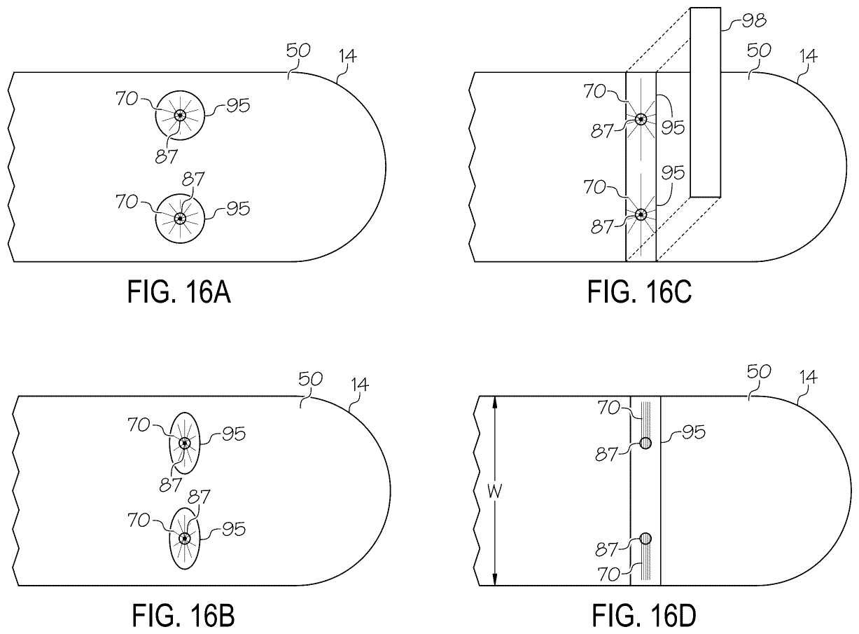

In one embodiment, recesses 95 may be of various shapes, sizes, and depths and be molded or otherwise formed within the top and bottom surfaces 50 and 32 of the plates 14 and 12. For example, as shown in FIG. 16A-C, the recesses 95 in the foot plate 12 and shank plate 14 may be circular, oval, or polygonal (such as a rectangular shaped channel). Specifically, in one example the recess(es) 95 in the shank plate 14 and foot plate 12 (not shown) are shaped as adjoining rectangular channels, as shown in FIG. 16C. In this embodiment, the channels 95 are configured to extend across a majority of, or substantially all of, or the entirety of the width W of the plates 12 (not shown) and 14. It should be appreciated that the recess(es) 95 in the foot plate 12 may be the same or different sizes and shapes as the recess(es) in the shank plate 14. As discussed above, the recesses 95 may be made in any size and shape suitable to at least accommodate the deflected end portions 90 and 92 of the fibers and resin.

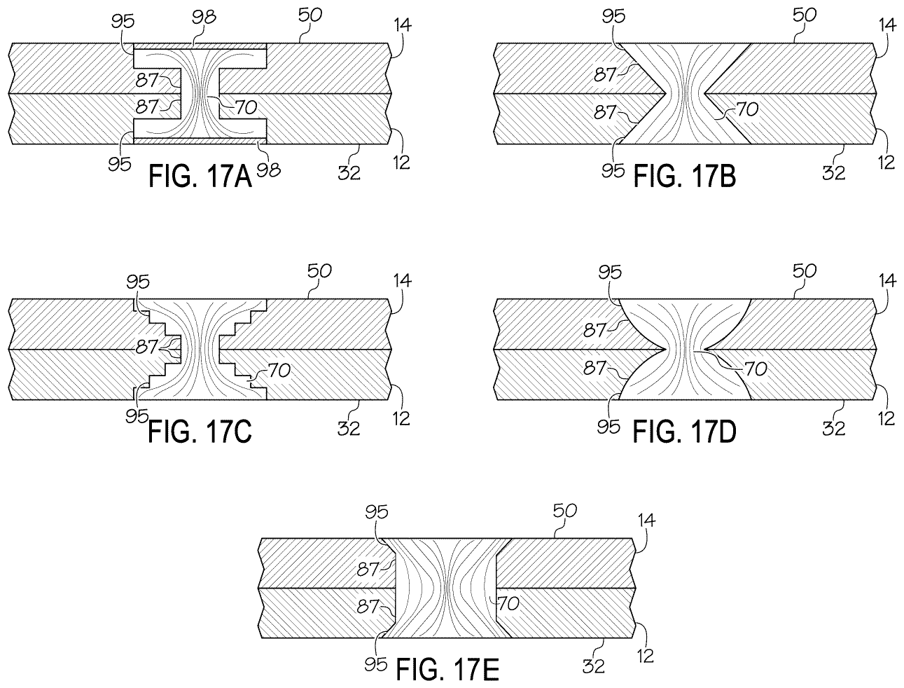

In one embodiment, as shown in FIGS. 16C and 17A, the recesses 95 collectively form a rectangular channel about ⅛″ deep, ½″ wide, and 2″ long (although it will be apparent that other sizes and relative dimensions may be used). In this embodiment, there are two composite attachment structures 70 disposed through two apertures 87 in the plates 12 and 14 configured to have adjoining recesses. As the ends of each of the composite attachment structures 70 are molded and deformed across the surface of the respective recesses 95, fibers within the structures 70 bend and/or deflect outwardly away from the apertures 87 within the recesses 95, while the resin surrounding the fibers fills the apertures 87 and at least partially or completely fills (or, potentially, overfills) the recesses 95 to form a strong bond with each of the plates 12 and 14. Optionally, the fibers may be folded over, unidirectionally (e.g., as shown in FIG. 16D) or multidirectionally (e.g., in both directions across the width W or flared or splayed radially as shown in FIGS. 16A-C), prior to molding. Optionally, the apertures 87 and recesses (channels) 95 could have a textured inner surface, such as a finely grooved, knurled, scuffed or other toothed surface (not specifically shown), to enhance bonding of the resin in the composite attachment structure 70 thereto. The texture could be achieved in any appropriate way, such as by molding, machining, sandblasting, etc.

In these and other such embodiments, the deflected end portions 90 and 92 of the composite attachment structure 70 may be capped by a separate layer of composite material 98 (see, e.g., FIGS. 16C and 17A), including reinforcing fibers (such as substantially linear or non-linear filament structures) and resin material, that may be lain into and molded to at least partially or completely fill (or, potentially, overfill) the respective recesses, and thereby further strengthen the bond between, and thus the structural integrity of, the attached plates 12 and 14. Additionally, capping layers of composite material 98 may be shaped to conform to the shape of the recess(es) 95 in the plates 12 and 14 and may help to provide visually smooth and aesthetic pleasing surfaces over the deflected end portions 90 and 92 of the composite attachment structure 70.

Optionally, additional operations could be implemented after initial molding to enhance the functionality, durability, or appearance of the composite attachment structure 70, such as buffing, machining, milling, dipping, spraying, decaling, coating, etc., so as to render the end portions 90, 92 of the composite attachment structure, or the capping layers 98 where present, flush with the adjoining top and bottom side surfaces 30, 32, 50, 52 of the plates 12 and 14, as applicable (see, e.g., FIGS. 16-25).

Referring now to FIGS. 17A-E, when plates 12 and 14 are adjacent one another, the recesses 95 and apertures 87 form an inner opening through the adjacent plates 12 and 14. The inner opening may be characterized by a centrally narrowed or narrowing cross-sectional shape, including, for example, bilaterally stepped (FIGS. 17A and 17C), tapered (FIG. 17B), helical, curved (FIG. 17D), and beveled (FIG. 17E) cross-sectional shapes and combinations thereof. Such inner openings, collectively formed by the recesses 95 and apertures 87, serve to strengthen the bond between, and thus the structural integrity of, the attached plates 12 and 14 by enabling the composite attachment structure 70 to be molded, formed, or otherwise configured to at least partially or completely fill (or, potentially, overfill) the inner opening, and thereby prevent or inhibit inadvertent separation of the plates 12 and 14.

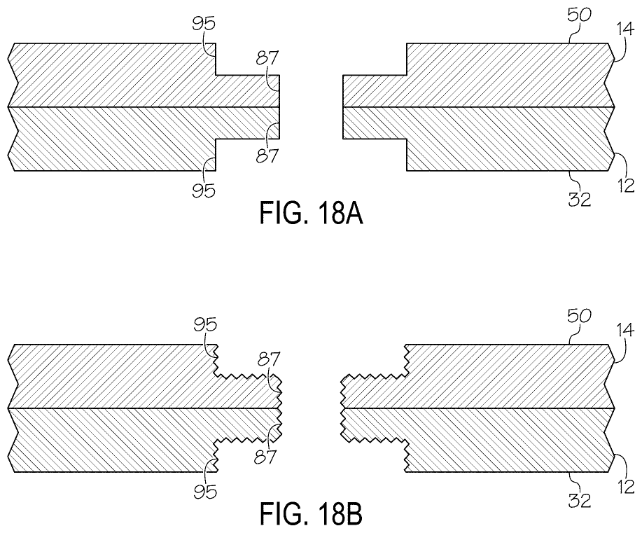

In another embodiment, the inner opening may be configured to have a textured inner surface such as a finely grooved, knurled, scuffed, or toothed surface (not specifically shown) to enhance bonding of the resin in the composite attachment structure 70 to the inner surfaces of the apertures 87 and recesses 95. Examples of non-textured and textured surfaces are shown in FIGS. 18A and 18B. The texture could be achieved in any appropriate way, such as by molding, machining, sandblasting, etc.

In yet another embodiment, the deflected top and bottom end portions 90 and 92 may be pressed onto the top and bottom surfaces 50 and 32 without the recesses 95. In these embodiments, the overlying and underlying layers 80A, 80B, respectively, of composite weave material may help to provide visually smooth and aesthetically pleasing surfaces over the deflected end portions 90 and 92 of the composite attachment structure 70.

Referring again to FIG. 2, the resin materials in the plates 12 and 14, the composite weave layers 80A, 80B, and the composite attachment structure 70 may be cured together against the mold parts 86A, 86B, and 86C. In an embodiment, a single curing process bonds all the adjoining composite materials together. This forms a seam 100 along which the shank plate 14 is bonded to the foot plate 12. The mold parts 86A-86C are shaped such that the seam 100 extends rearward from the forward ends 28, 56 of the plates 12 and 14 until the forward end of a layer 82A of peel ply film blocks the formation of a bond where it extends between the shank plate 14 and the foot plate 12.

The layers 82B and/or 82A of peel ply film optionally are removed to expose a gap 105 (FIG. 4) across which the bottom surface 52 of the shank plate 14 is spaced apart from the top surface 30 of the foot plate 12. The gap 105 enables the shank plate 14 and the foot plate 12 to deflect independently of one another at that location. The optional removal of the layer 82D of peel ply film does not affect the mechanical characteristics of the prosthetic foot structure 10 and improves the cosmetic appearance of the final part.

During use of the composite prosthetic foot structure 10 by a wearer, when the wearer places their weight on the prosthetic structure 10 and removes their weight in, for example, a walking gait, the foot plate 12 and shank plate 14 deflect toward and away from each other across the gap 105. Stresses can concentrate at the rear end 106 (FIG. 4) of the seam 100 where the foot plate 12 and shank plate 14 cannot flex independently. However, the composite attachment structure 70 is located to bear the load of these stresses to prevent separation of the plates 12 and 14 at the seam 100. Specifically, if the seam 100 were extended to reach rearward past the composite attachment structure 70, a rearward extending section of the seam 100 might rupture as the foot plate 12 and shank plate 14 repeatedly deflect away from one another during use.

For this reason, in certain embodiments the composite attachment structure 70 is located at least partially to the rear of the seam 100 so that the seam 100 does not reach rearward (to the left in FIGS. 4 and 5) past the composite attachment structure 70. In the embodiment shown in FIGS. 1 and 2, the composite attachment structure 70 is located entirely to the rear of the seam 100, so that the gap 105 is present on both sides of the composite attachment structure 70. In the embodiment shown in FIG. 5, the composite attachment structure 70 is located partially within, and extends through the gap 105 and is located partially within and extends through the seam 100, so that the rear end 106 of the seam is located at the composite attachment structure 70. In each of these exemplary embodiments, the fibers in the composite attachment structure 70 are primarily aligned unidirectionally with the transverse axis 75 along their lengths reaching through the foot plate 12, the gap 105, the shank plate 14, and for the embodiment of FIG. 5, through the seam 100.

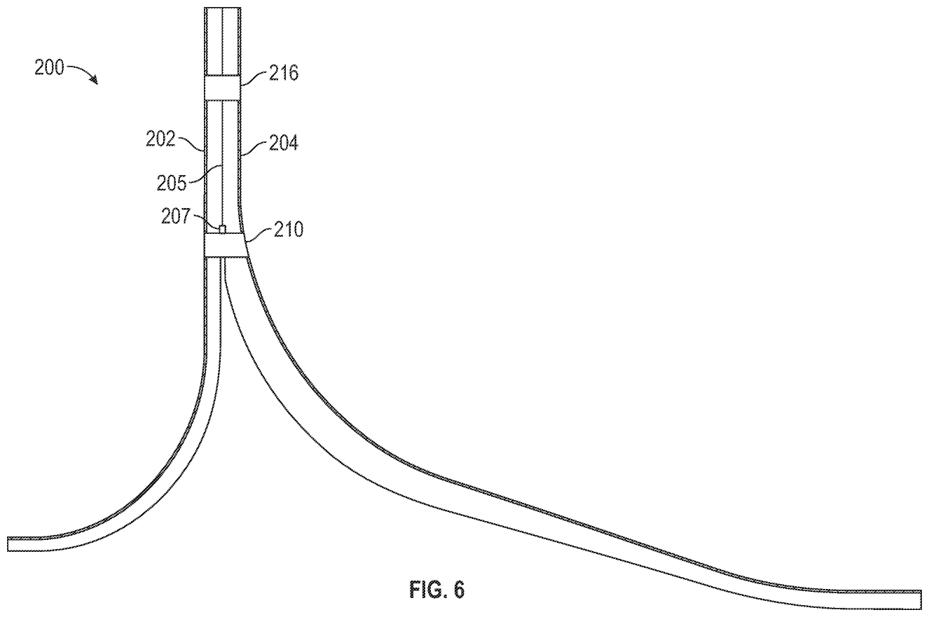

In the alternative embodiment of FIG. 6, a plate assembly in a prosthetic foot structure 200 includes first and second plates 202 and 204. These plates 202 and 204 have composite compositions substantially the same as the composite compositions of the plates 12 and 14 described above. The plates 202 and 204 are joined along a seam 205 that terminates at a gap 207. The foot structure 200 further includes a composite attachment structure 210 having substantially the same composition and structural configuration as the composite attachment structure 70 described above. The composite attachment structure 210 thus reaches through the plates 202 and 204 and across the gap 207 to prevent separation of the plates 202 and 204 at the seam 207. One or more additional composite attachment structures 216 may also be provided.

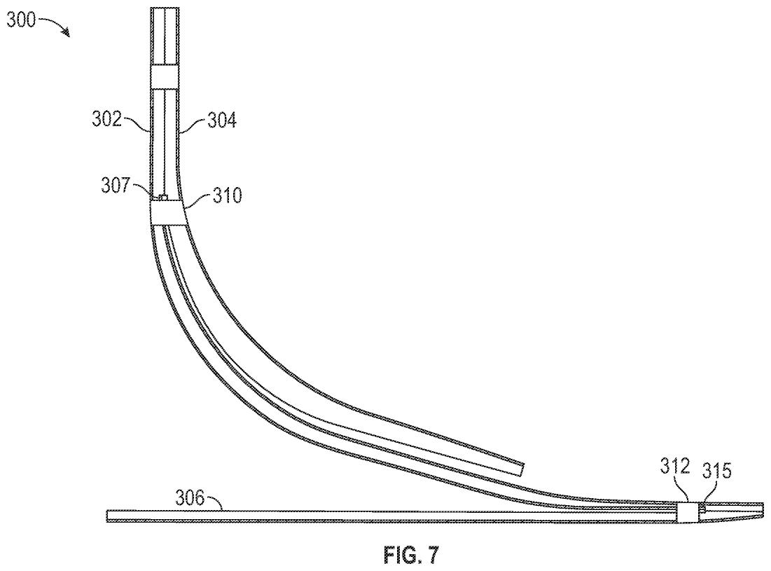

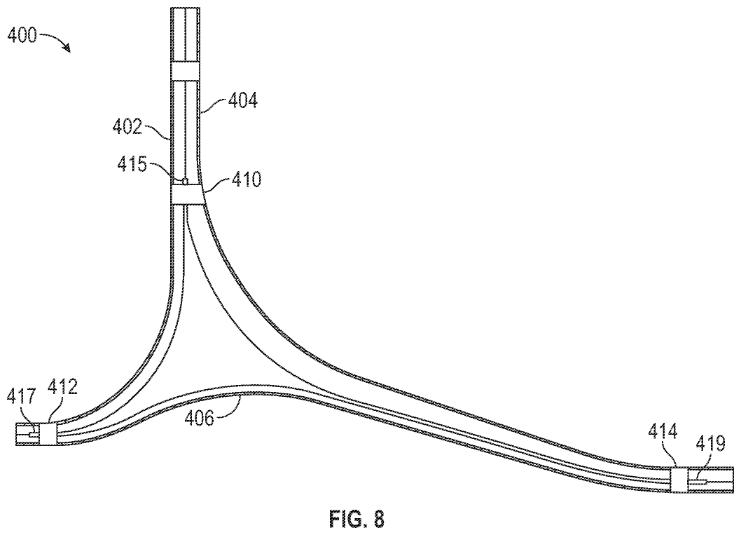

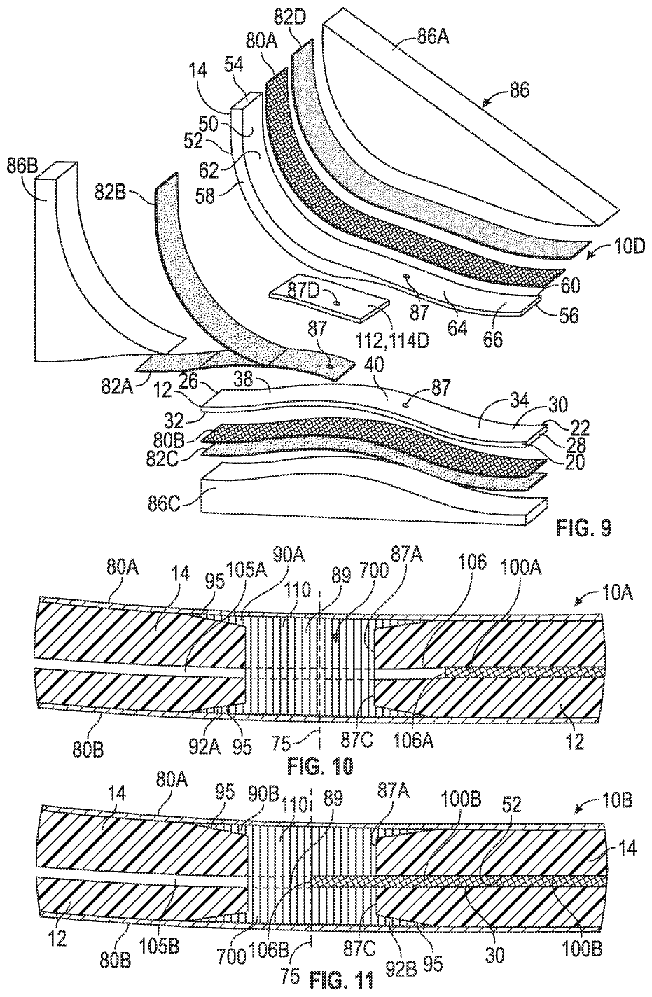

Second and third alternative embodiments are shown in FIGS. 7 and 8, respectively. In the embodiment of FIG. 7, the foot structure 300 includes three composite plates 302, 304, and 306 attached together by two composite attachment structures 310 and 312. One of these composite attachment structures 310 reaches through the plates 302, 304 and a gap 307 between the plates 302, 304. The other composite attachment structure reaches through the plates 302, 306 and a gap 315 between those plates 302, 306. In the embodiment of FIG. 8, the foot structure 400 similarly includes three composite plates 402, 404, and 406 with three composite attachment structures 410, 412 and 414 at respective gaps 415, 417, and 419. One or more additional composite attachment structures also may be provided in the embodiments of FIGS. 7 and 8, as shown.

As shown in FIG. 10, a fourth embodiment of the composite prosthetic foot structure, generally designated 10A, is identical to the embodiment of FIGS. 1 and 4, except as follows. The attachment structure 700 is made up of fibers 110 without a supporting matrix of a resin. In an embodiment, the fibers are oriented as shown in FIG. 10 to extend through openings 87A, 87C of the shank plate 14 and foot plate 12, respectively, and across the gap 105A. Thus, the prosthetic foot 10A includes a foot plate 12 having a toe portion 34 (FIG. 1) and a heel portion 38. The shank plate 14 is bonded to the foot plate 12 along a seam 100A that extends from the toe portion 34 rearward toward the heel portion 38 to terminate at an end 106A. The shank plate 14 is spaced apart from the foot plate 12 forming a gap 105A between the foot plate and shank plate that extends rearward from the end 106A of the seam.

An attachment structure 700 attaches the foot plate 12 to the shank plate 14. As shown in FIG. 10, the attachment structure 700 includes fibers 110, and consists or consists essentially of fibers 110, extending through the shank plate 14, the gap 105A, and the foot plate 12. In the embodiment of the prosthetic foot 10A of FIG. 10, the fibers 110 of the attachment structure 700 extend through the gap 105A but not through the seam 100A. The end 106A of the seam 100A terminates along the lengths of the shank plate 14 and foot plate 12 forward of the attachment structure 700.

A fifth embodiment of the prosthetic foot system, generally designated 10B and shown in FIG. 11, is identical to the embodiments 10, 10A of the prosthetic foot systems shown in FIGS. 1 and 10, respectively, except as follows. The end 106B of the seam 100B extends from the toe portion 34 of the foot plate 12 (FIG. 1) rearward between, and joins, the top surface 30 of the foot plate and bottom surface 52 of the shank plate 14 to terminate at a point along the length of the shank plate that coincides with the location of the attachment structure 700, so that the end 106B of the seam 100B is transversely aligned along a length of the prosthetic foot 10B with the composite attachment structure. With the embodiment of the prosthetic foot 10B of FIG. 11, the fibers 110 of the attachment structure 700 extend through the gap 105B and also extend through the seam 100B. The top end portion 90B and bottom end portion 92B of the fibers 110 are bent or flare radially outward from the transverse axis 75 of the cylindrical central shaft 89 of the composite attachment structure.

An exemplary embodiment of the prosthetic foot, generally designated 10B, is shown in FIG. 11. In this embodiment, the end 106B of the seam 100B coincides with, or approximately with, a midpoint of the attachment structure 700, coinciding with, or approximately with, the transverse axis 75. In other embodiments, the end 106B of the seam 100B terminates coinciding transversely with a portion of the attachment structure 700 forward toward the toe portion 34 of the transverse axis 75 or coinciding transversely with a portion of the attachment structure 700 rearward of the transverse axis 75 toward the heel portion 38.

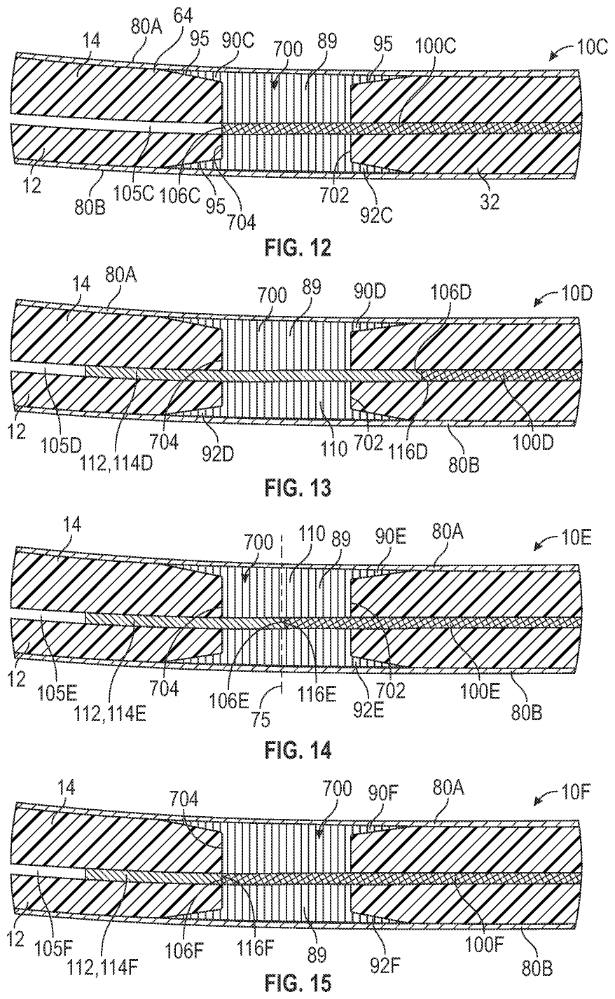

In the embodiment of the prosthetic foot, generally designated 10C, shown in FIG. 12, the cylindrical central shaft 89 of the attachment structure 700 has a forwardmost leading edge 702 toward the toe portion 34 (FIG. 1), a rearmost trailing edge 704 toward the heel portion 38, and the cylindrical shaft 89 extends through the shank plate 14, the seam 100C, and the foot plate 12. The flared top portion 90C overlies the top surface 64 of the shank plate 14, and the flared bottom portion 92C overlies a bottom surface 32 of the foot plate 12. The end 106C of the seam 100C is transversely aligned along a length of the prosthetic foot between the leading edge 702 and the trailing edge 704. In the exemplary embodiment shown, the end 106C of the seam 100C coincides longitudinally with, or approximately with, but not rearward of, the trailing edge 704 of the cylindrical central shaft 89 of the attachment structure 700.

In an embodiment of the prosthetic foot, generally designated 10D, shown in FIGS. 9 and 13, a flexible material 112 is placed in the gap 105D formed between the shank plate 14 and the foot plate 12. As shown in FIG. 9, in embodiments the flexible material may take the form of a strip 114D that extends a width of the foot plate 12 and the matching width of the shank plate 14. In embodiments, the flexible material 112 is selected from natural rubber, synthetic rubber, nitrile rubber, silicone rubber, a urethane rubber, chloroprene rubber, ethylene rubber, and vinyl acetate rubber.

In the exemplary embodiment of FIG. 13, the strip 114D of flexible material 112 extends along the gap 105D from a point forward of the leading edge 702 of the cylindrical central shaft 89 of the attachment structure 700 toward the toe portion 34 (FIG. 1) of the prosthetic foot 10D rearward of the attachment structure 700 toward the heel portion 38. In an embodiment, the flexible material 112 fills the gap 105D adjacent and, in embodiments, contacting the end 106D of the seam 100D. The strip 114D extends rearward of the end 106D to terminate at a point past and rearward of the trailing edge 704 of the cylindrical central shaft 89 of the attachment structure 700.

In this embodiment 10D, the flexible material 112 contacts the end 106D of the seam 100D forward of the attachment 700 structure toward the toe portion 34 such that the fibers 110 extend through the strip 114D of flexible material 112 in the gap 105D. As shown in FIG. 9, in embodiments, the strip 114D includes an aperture 87D through which the fibers 110 of the attachment structure 700 extend.

As shown in FIG. 14, in an embodiment of the prosthetic foot, generally designated 10E, the end 106E of the seam 100E is transversely aligned along a length of the prosthetic foot with the attachment structure 700 and the flexible material 112 contacts the end of the seam such that the fibers 110 extend through the flexible material in the gap 105E and through the seam 100E. In embodiment shown in FIG. 14, the end 106E coincides or approximately coincides with the transverse axis 75, so that the forward end 116E of the flexible strip 114E meet at or approximately at the transverse axis 75. In other embodiments, the forward end 116E and the end 106E meet between the transverse axis 75 and the leading edge 702 of the cylindrical central shaft 89 or between the transverse axis and the trailing edge 704.

In another embodiment of the prosthetic foot, generally designated 10F, shown in FIG. 15, The seam 100F extends from the toe portion 34 rearward and terminates so that the end 106F of the seam coincides longitudinally with the trailing edge 704 of the cylindrical central shaft 89 of the attachment structure 700. The strip 114F of flexible material 114 fills the gap 105F between the shank plate 14 and the foot plate 12 such that the forward end 116F is adjacent and abuts the end 106Fof the seam 100F at the trailing edge 704.

As shown in FIG. 13-15, the performance characteristics of the prosthetic foot 10D-10F, such as the compressibility of the shank plate 14 against the foot plate 12 and the resiliency of the shank plate and foot plate engagement, can be varied by varying the length and placement of the flexible material 112 within the gap 105D-105F rearward of the seam 100D-100F. In each embodiment of the prosthetic foot 10D-10F, the flexible material 112 completely fills and extends along the gap 105D-105F for a selected distance rearward of the attachment structure 700 toward the heel portion 38 of the shank plate 12. In embodiments, the strip 114D-114F extends selected distances along the gap 105D-105F up to the point rearward of the attachment structure 700 where the shank plate 14 and the foot plate 12 do not deflect to contact each other during use. And in embodiments, the flexible material 112 fills the gap 105D-105F adjacent and contacting the end 106D-106F of the seam 100D-100F.

In the prosthetic foot 10D of the embodiment of FIG. 13, the flexible material 112 contacts the end 106D of the seam 100D forward of the attachment structure 700 toward the toe portion 34 such that the fibers 110 extend through the flexible material in the gap 105D. In the prosthetic foot 10E of the embodiment of FIG. 14, the end 106E of the seam 100E is transversely aligned along a length of the prosthetic foot with the attachment structure 700 and the flexible material 112 contacts the end of the seam such that the fibers 110 extend through both the flexible material in the gap 105E and through the seam. In a particular embodiment, the flexible material 112 and the seam 110E abut at or at approximately the transverse axis 75, and in other embodiments forward or rearward of it. Thus, in the embodiments 10D and 10E of the prosthetic foot depicted in FIGS. 13 and 14, the flexible material 112 extends rearward from the end 106D, 106E of the seam 100D, 100E such that the attachment structure 700 passes through the flexible material.

In the embodiment of the prosthetic foot 10F of FIG. 15, the end 106F of the seam 100F transversely coincides with a rearmost trailing edge 704 of the attachment structure 700 toward the heel portion 38, and the flexible material 112 fills the gap 105F adjacent and contacting the end of the seam and extends rearward toward the heel portion 38.

In embodiments, the length of the strip 114D-114F of flexible material, the composition of the flexible material, and the of the forward end 116D-116F along the length of the prosthetic foot are selected to vary the resiliency and flexure characteristics of the prosthetic foot 10D-10F. The location of the strip 114D - 114F also provides a reduction in the noise that might otherwise result from contact between the shank plate 14 and foot plate 12 during walking by a user.

In each of the embodiments 10D-10F of the prosthetic foot, individual ones of the fibers 110 are oriented to extend longitudinally and continuously through the shank plate 14, the gap 105A-105F, the seam 110A-110F and/or the flexible strip 114D-114F, and the foot plate 12. In embodiments, the fibers 110A-110F are made of a material selected from one or more of steel, carbon, glass, nylon, an aramid, and polyethylene.

In embodiments, the fibers 110 are angled radially outward from the cylindrical central shaft 89 to form the flared top portion 90A-90F and the flared bottom portion 92A-92F of the attachment portion 700. In embodiments, flared top portion 90A-90F and the flared bottom portion 92A-92F of the attachment portion 700 are positioned within recesses 95 formed in the top surface 50 and bottom surface 32 of the shank portion 14 and foot portion 12, respectively. In embodiments, the entire top surface 50 and bottom surface 32, including the flared top portion 90A-90F and the flared bottom portion 92A-92F, are covered by the layers 80A, 80B, respectively, of composite weave material.

In sum, the exemplary embodiments 10A-10F of the disclosed prosthetic foot shown in FIG. 9-15 comprise: a foot plate 12 having a toe portion 34 and a heel portion 38 and a shank plate 14 bonded to the foot plate along a seam 100A-100F that extends from the toe portion rearward toward the heel portion to terminate at an end 106A-106F. The shank plate 14 is spaced apart from the foot plate 12 forming a gap 105A-105F between the foot plate and shank plate extending rearward from the end 106A-106F of the seam. An attachment structure 700 attaches the foot plate 12 to the shank plate 14, wherein the composite attachment structure includes a central shaft 89 of fibers 110 extending through the shank plate, the seam (in the embodiments of FIGS. 9, 10, 11, 12, 14, and 15), and the foot plate. An end 106A-106F of the seam 100A-100F is transversely aligned along a length of the prosthetic foot 10A-10F with a rearmost trailing edge 704 of the cylindrical central shaft 89.

In embodiments, the prosthetic foot 10D-10F further comprises a flexible material 112 in the gap 105D-105F contacting and extending rearward from the trailing edge 106D-106F of the seam 100D-100F. In embodiments, the foot plate 12 and the shank plate 14 each are discrete plates made of a composite, such as fiber reinforced resin or carbon fiber reinforced plastic (CFRP).

An exemplary embodiment of a method of making a prosthetic foot is shown in FIG. 9 with reference to the embodiment 10D of the prosthetic foot shown in FIG. 13. The method includes placing a first layer 82C of peel ply material on a sole mold part 86C, placing a first layer 80B of composite weave on the first layer of peel ply material, and placing a foot plate 12 on the first layer of composite weave, the foot plate having a toe portion 34 and a heel portion 38. Second and third layers 82A, 82B, respectively of peel ply material are placed over and under a heel mold part 86B. A strip 114D of flexible material 112 is placed over a portion of the second layer 82A of peel ply material and a shank plate 14 is placed over the portion of the second layer 82A of peel ply material and the strip 114D of flexible material 112.

An attachment structure 700 is inserted through the shank plate 14, the strip 114D of flexible material 112, the portion of the second layer 82A of the peel ply material, and the foot plate 12. The attachment structure 700 includes fibers 110 extending through the shank plate 14, the flexible material 112, and the foot plate 12. A second layer 80A of composite weave is placed on a top surface 50 of the shank plate14, and a fourth layer 82D of peel ply material is placed over the second layer 80A of composite weave. An upper mold part 86A is placed over the fourth layer 82D of peel ply material.

The first layer 82C of peel ply material, the first layer 80B of composite weave, the foot plate 12, second and third layers 82A, 82B, respectively, of peel ply material, the strip 114D of flexible material 112, the shank plate 14, the second layer 80A of composite weave, the fourth layer 82D of peel ply material, and the attachment structure 700 are compressed between the upper mold part 86A, the heel mold part 86B, and the sole mold part 86C to bond the shank plate 14 to the foot plate 12 to form a seam 100D therebetween that extends forward of the attachment structure 700 toward the toe portion 34. The attachment structure 700 includes fibers 110 extending through the shank plate 14, the strip 114D of flexible material 112, and the foot plate 12, thereby forming a flared top portion 90D that overlies a top surface 50 of the shank plate and a flared bottom portion 92D that overlies a bottom surface 32 of the foot plate 12 and also forms a gap 105D between the shank plate and the foot plate that extends rearward of the flexible strip 114D toward the heel portion 38.

The various embodiments 10, 10A-10F of the prosthetic foot described above and illustrated in the Figures provide a rugged, resilient, and yet lightweight prosthetic foot, the components of which can be customized easily to suit the preferences of a user regarding resiliency, flexure, and feel. The inclusion of the described attachment structure 700 made of elongate fibers 110 secures the shank plate 14 to the foot plate 12 and prevents failure of the seam 100, 100A-100F, yet does not restrict the flexibility of the overall foot 10, 10A-10F.

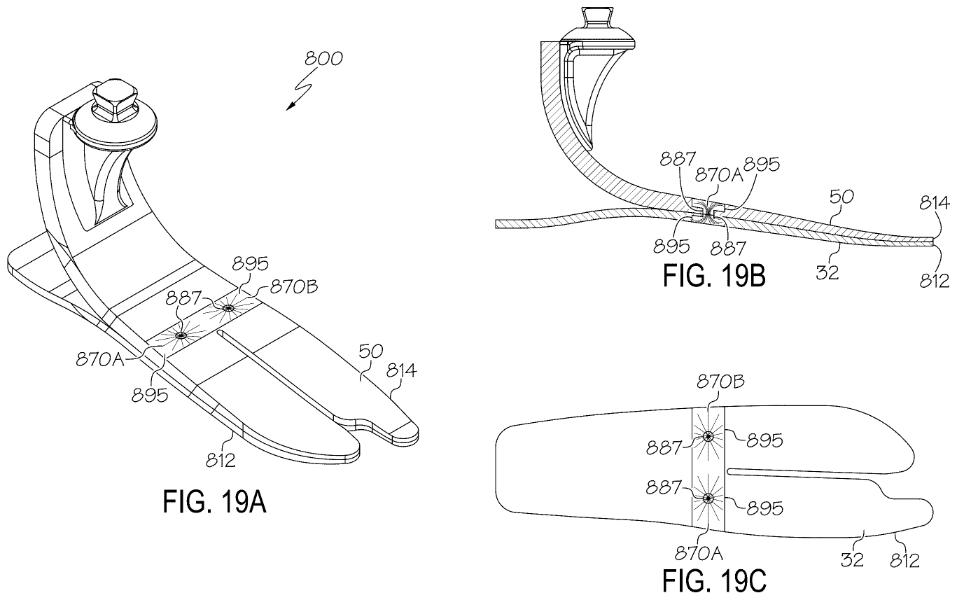

In an exemplary embodiment, shown in FIGS. 19A-C, the foot structure 800 includes a composite foot plate 812 configured to be attached to a composite shank plate 814 by first and second composite attachment structures 870A and 870B. The composite foot plate 812 and composite shank plate 814 may be pre-molded with recesses 895 formed on the bottom side and top side surfaces 32, 50, respectively. Although illustrated in FIGS. 19A-C as single recesses 895 including multiple apertures 887, recesses 895 could be formed as separate recesses 895 each formed about one such aperture 887. Additionally, it will be appreciated that one or more recesses 895 in the foot plate 812 may have a first plurality of apertures 887 and one or more recesses 895 in the shank plate 814 may have a second plurality of apertures 887 aligned with the first plurality of apertures 887. In the embodiment of FIGS. 19A-C, the composite attachment structures 870A and 870B (comprising, for example, reinforcement fibers embedded or otherwise disposed in a resin material) are configured to be disposed through apertures 887 in the shank plate 814 and corresponding apertures 887 in the foot plate 812 with the ends of the composite attachment structures 870A and 870B folded (unidirectionally or multidirectionally (e.g., flared or splayed radially)) over the top surfaces of the recesses 895 of the shank plate 814 and of the foot plate 812. The ends of the composite attachment structures 870A and 870B may at least partially or completely fill (or, potentially, overfill) the recesses 895 or, as described previously, a capping layer of composite material (not shown) may be shaped to conform to the shape of the recesses 895 in the shank plate 814 and foot plate 812 to at least partially or completely fill (or, potentially, overfill) the recesses 895, and may help to provide visually smooth and aesthetically pleasing surfaces over the deflected end portions of the composite attachment structures 870A and 870B.

Optionally, additional operations could be implemented after initial molding to enhance the functionality, durability, or appearance of the composite attachment structures 870A and 870B, such as buffing, machining, milling, dipping, spraying, decaling, coating, etc., so as to render some or all of the end portions of the composite attachment structures 870A and 870B, or the capping layers where present, flush with the adjoining bottom side and top side surfaces 32, 50 of the plates 812 and 814.

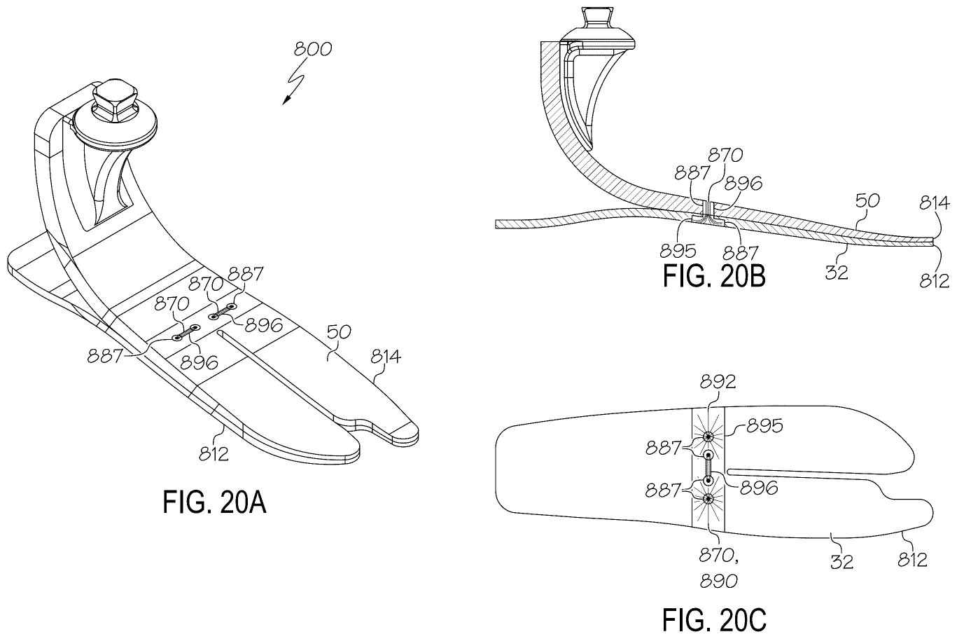

In another embodiment, as shown in FIGS. 20A-C, the foot structure 800 includes a composite foot plate 812 configured to be attached to a composite shank plate 814 by a composite attachment structure 870 that is woven through a plurality of apertures 887. In this embodiment, the composite foot plate 812 and composite shank plate 814 are pre-molded with recesses 895 on the outside surfaces to accommodate the composite attachment structure 870. It will be appreciated that one or more recesses 895 in the foot plate 812 may have a first plurality of apertures 887 and one or more recesses 895 in the shank plate 814 may have a second plurality of apertures 887 aligned with the first plurality of apertures 887. Although illustrated in FIGS. 20A-C as a single recess 895 in the top side surface 50 of the shank plate 814, recesses 895 could be formed as separate recesses 895 each formed about a plurality of apertures 887. As shown in FIG. 20C, the recesses 895 may include a rectangular channel, disposed on the bottom surface of the foot plate 812, and one or more grooves 896, disposed on the top surface of the shank plate 814 for receiving intermediate portions of a plurality of elongated fibers (together with the resin material in which such fibers are embedded or otherwise disposed) configured to extend continuously between and through the apertures 887. It will be appreciated that the rectangular channel may itself include a groove 896, depending upon the thickness of the plurality of elongated fibers, and that the rectangular channels themselves may form a groove or be formed in lieu of a groove, and further that in variations of the embodiment the positions of the illustrated rectangular channel(s) and the groove(s) may be reversed with respect to the composite foot plate 812 and the composite shank plate 814. An end portion 890 of the composite attachment structure 870 is disposed in a recess, e.g., the rectangular channel as shown, and the plurality of fibers pass through the apertures 887 in the foot plate 812 and the shank plate 814, with the other end portion 892 of the composite attachment structure 870 disposed in a recess, e.g., the rectangular channel as shown. More specifically, in the exemplary embodiment shown, the plurality of fibers pass through the foot plate 812 and the shank plate 814 through an opposing pair of apertures 887, cross the top of the shank plate 814, reach back through the foot plate 812 and shank plate 814 through another opposing pair of apertures 887, cross the bottom of the foot plate 812, reach back through the foot plate 812 and the shank plate 814 through yet another pair of opposing apertures 887, cross the top of the shank plate 814, and reach back through the foot plate 812 and shank plate 814 through a further pair of opposing apertures 887, with the ends 890, 892 of the attachment structure 870 positioned in the composite foot plate 812 so that the ends 890, 892 of the composite attachment structure 870, including folded over fibers (together with the resin material in which such fibers are embedded or otherwise disposed) as described previously, fit within and at least partially or completely fill (or, potentially, overfill) the recesses 895, when molded into place. Again, it will be appreciated that in variations of the embodiment the positions of the ends 890, 892 of the composite attachment structure 870 and the specific crossings of the plates 812, 814 by portions of the composite attachment structure 870 may be reversed with respect to the composite foot plate 812 and the composite shank plate 814, and that the end portion 892 of the composite attachment structure 870 does not need to be disposed in a recess 895 on the same plate, 812 or 814, as the end portion 890 of the composite attachment structure 870. In variations of the embodiment, the plurality of elongated fibers of the attachment structure 870 may be a spun yarn or a thin strip of prepreg material that is stitched through the plates before or after the plates are molded. In further variations of the embodiment, one or more of the recesses 895 (e.g., recesses 895 in the shank plate 814 and foot plate 812, or the recesses 895 in the shank plate 814, or the recesses 895 in the foot plate 812) may be capped by a separate layer of composite material (not specifically shown) including reinforcing fibers (such as linear, substantially linear, or non-linear filament structures) and resin material, that may be lain into and molded, formed, or otherwise configured to at least partially or completely fill (or, potentially, overfill) the respective recesses.

Optionally, additional operations could be implemented after initial molding to enhance the functionality, durability, or appearance of the woven composite attachment structure 870, such as buffing, machining, milling, dipping, spraying, decaling, coating, etc., so as to render the end portions 890, 892 of the composite attachment structure 870, or the capping layers where present, flush with the adjoining bottom side and top side surfaces 32, 50 of the plates 812 and 814. Likewise, such operations may render the connecting portions of the plurality of elongated fibers that extend between apertures 887, or the capping layers where present, flush with the adjoining bottom side and top side surfaces 32, 50 of the plates 812 and 814.

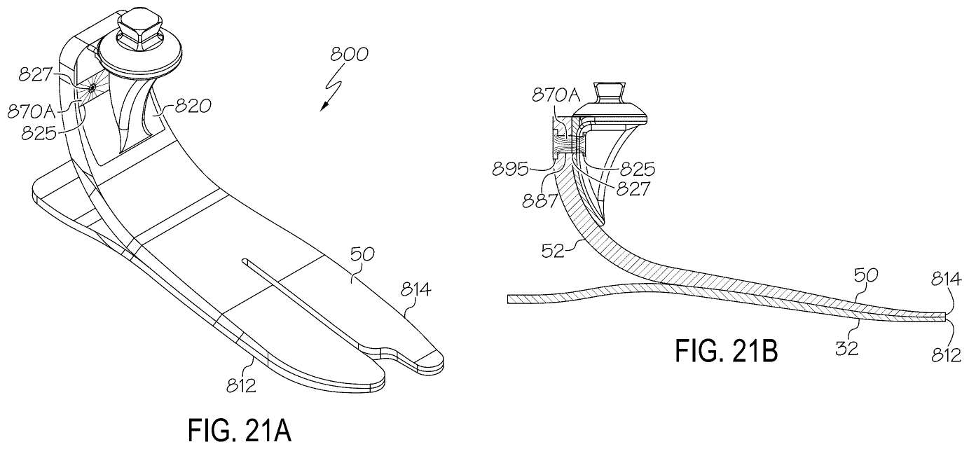

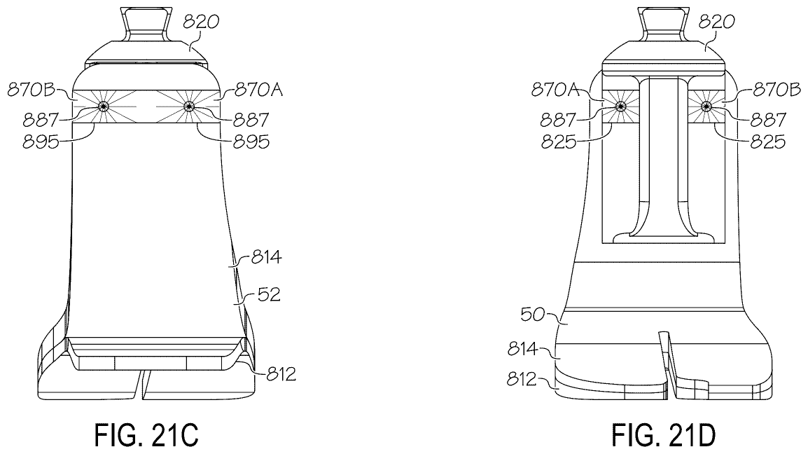

In yet another embodiment, as shown in FIGS. 21A-D, a foot structure 800 may further include a composite shank plate 814 that is configured to be attached to an adapter 820. The adapter 820 may be any structure capable of attaching the foot structures disclosed herein at the upper portion of the shank plate 814 with another component of a prosthetic leg, such as a socket or a knee joint (not shown). The adapter 820 may be attached to the shank plate 814 using a composite attachment structure 870, or by two composite attachment structures 870A and 870B. The composite attachment structures 870, 870A, and 870B may be constructed in the same or similar manner as the attachment structures 70 discussed above, including a plurality of fibers embedded or otherwise disposed in a resin material. In the illustrated embodiment, the composite attachment structures 870 are configured to be disposed through an aperture 887 in the upper portion 862 of the shank plate 814 and through an aperture 827 in the adapter 820. The composite shank plate 814 and adapter 820 may be further configured to include recesses 895, 825, similar to those described above, that are capable of accommodating enlarged end portions 890 and 892 of the attachment structures 870 that have been folded (unidirectionally or multidirectionally (e.g., flared or splayed radially)) over the top surfaces of recesses 895, 825 of the shank plate 814 and of the adapter 820, respectively. The ends of the composite attachment structures 870A and 870B may at least partially or completely fill (or, potentially, overfill) the recesses 895, 825 or, as described previously, a capping layer of composite material (not shown) may be shaped to conform to the shape of the recesses 895 in the shank plate 814 and the recesses 825 in the adapter 820 to at least partially or completely fill (or, potentially, overfill) the recesses 895, 825, and may help to provide visually smooth and aesthetically pleasing surfaces over the deflected end portions of the composite attachment structures 870A and 870B.

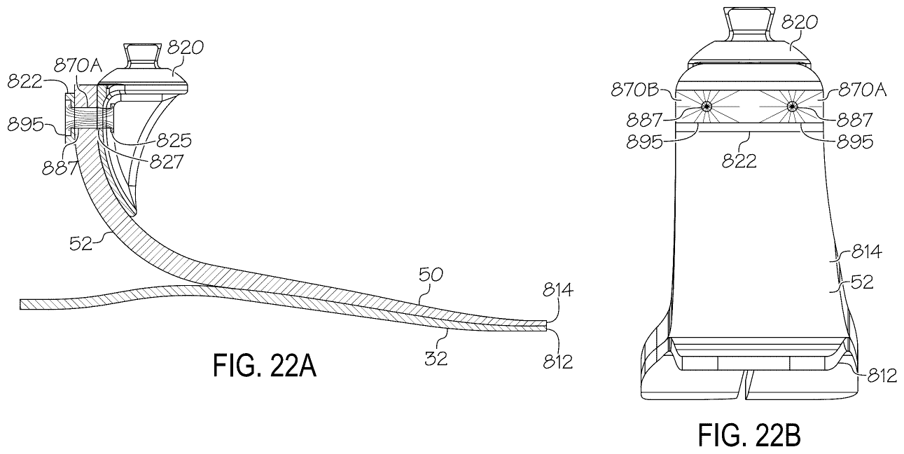

As shown in FIGS. 22A and 22B, the foot structure 800 may further include an optional backing plate 822 disposed along a back side of the shank plate 814. In such embodiments, the attachment structures 870 may be configured to extend through the backing plate 822, the shank plate 814, and the aperture 827 in the adapter 820. The backing plate 822 and the adapter 820 may be configured to include recesses 895, 825, similar to those described above, to accommodate the folded over end portions of the attachment structures 870A and 870B and any capping layers, as previously described above.

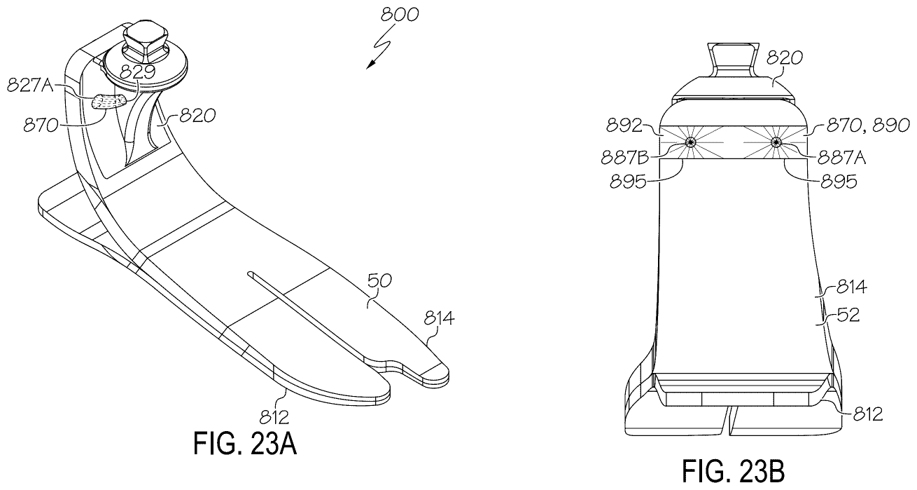

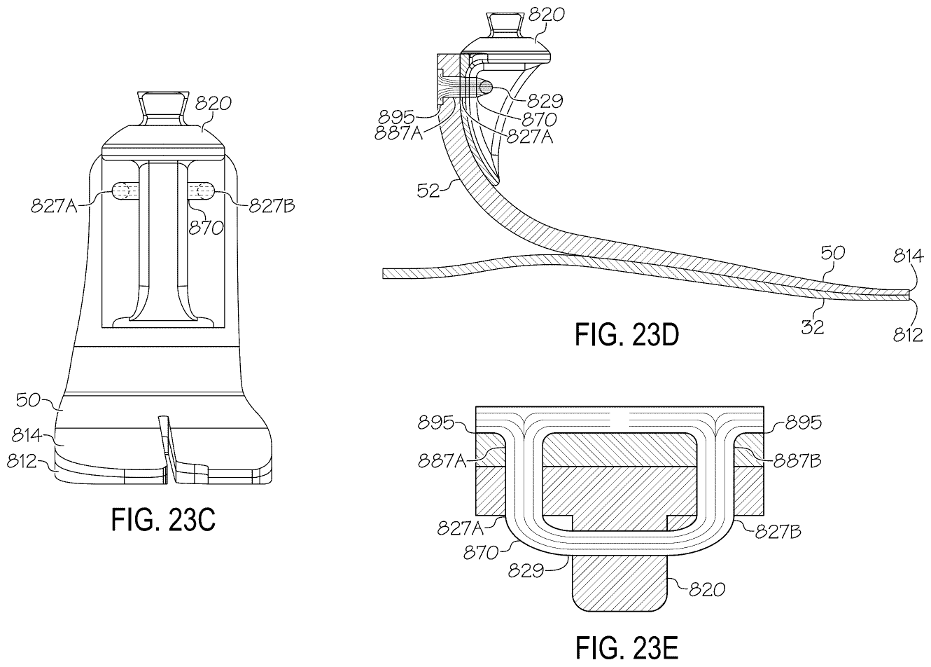

In another embodiment, as shown in FIGS. 23A-E, the foot structure 800 includes a composite shank plate 814 attached to an adapter 820 using a U-shaped composite attachment structure 870 (best seen in cross-sectional view in FIG. 23E). In this embodiment, the composite attachment structure 870 is configured to reach through a first aperture 887A in the shank plate 814, a corresponding first aperture 827A in the adapter 820 disposed on one side of the adapter, through an aperture 829 in the central body of the adapter 820, back through a second aperture 827B in the adapter 820 on the opposite side of the adapter 820, and back through a corresponding second aperture 887B in the shank plate 814. The composite shank plate 814 may include recesses 895, similar to those described above, to accommodate the folded over end portions 890, 892 of the attachment structure 870 and any capping layers, as previously described above.

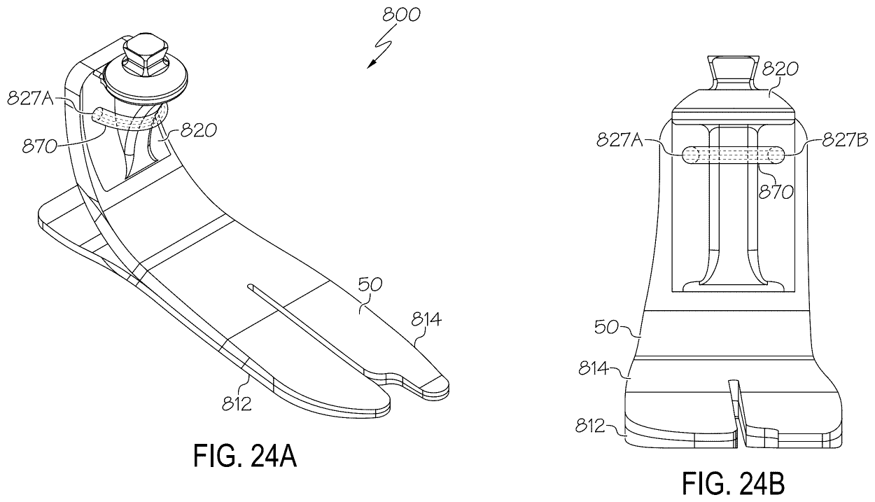

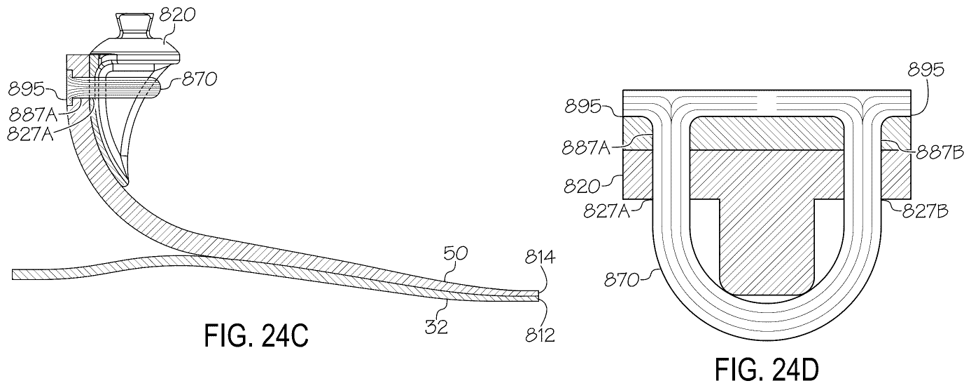

In another embodiment, as shown in FIGS. 24A-D, the foot structure 800 includes a composite shank plate 814 attached to another adapter 820 using a U-shaped composite attachment structure 870. In this embodiment, the composite attachment structure 870 is configured to reach through a first aperture 887A in the shank plate 814, a corresponding first aperture 827A in the adapter 820 disposed on one side of the adapter, around the exterior of the central body of the adapter 820, back through a second aperture 827B in the adapter 820 on the opposite side of the adapter 820, and back through a corresponding second aperture 887B in the shank plate 814. The composite shank plate 814 may include recesses 895, similar to those described above, to accommodate the folded over end portions 890, 892 of the attachment structure 870 and any capping layers, as previously described above.

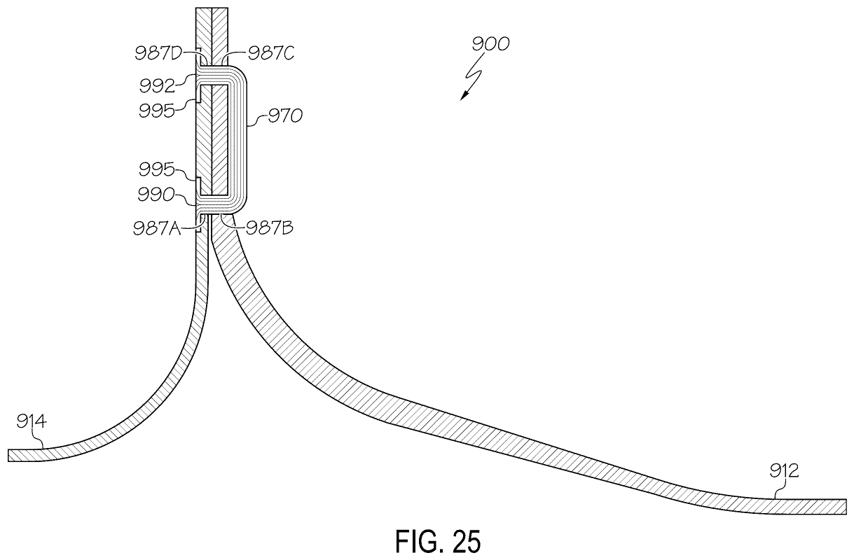

As shown in FIG. 25, the foot structure 900 may include a composite toe-shank plate 912 configured to be attached to a composite heel-shank plate 914 by a U-shaped composite attachment structure 970. These plates 912 and 914 have composite compositions substantially the same as the composite compositions of the plates 12 and 14 described above, and the U-shaped composite attachment structure 970 may have composite composition and end portion configurations to those of the composite attachment structures 70 and 870 described above. The U-shaped attachment structure 970 may be configured to have a first end portion 990 extending through at least a first aperture 987A in the heel-shank plate 914 and a corresponding first aperture 987B in the toe-shank plate 912, a connecting portion crossing the front of the toe-shank plate 912, and a second end portion 992 reaching back through at least a second aperture 987C in the toe-shank plate 912 and a second aperture 987D in the heel-shank plate 914. The composite heel-shank plate 914 may include recesses 995 along its back surface, similar to those described above, to accommodate the folded over end portions 990 and 992 of the attachment structure 970. The deflected end portions 990, 992 of the attachment structure 970 may be molded, formed, or otherwise configured to at least partially or completely fill (or, potentially, overfill) the recessed surface of the recesses 995 to form a strong bond with each of the plates 912 and 914. Optionally, the deflected end portions 990 and 992 of the composite attachment structure 970 may be capped by a separate layer of composite material (not shown) that may be lain into and molded, formed, or otherwise configured to at least partially or completely fill (or, potentially, overfill) the respective recesses.

While the forms of apparatuses and methods herein described constitute preferred and alternate embodiments of the disclosed composite prosthetic foot structure, it is to be understood that the invention is not limited to these precise forms of apparatuses and methods, and that changes may be made thereto without departing from the scope of the invention.

What is claimed is:

1. A composite prosthetic foot assembly, comprising:
a composite foot plate having a toe portion, a heel portion, an upper surface, a lower surface, at least one aperture extending through the foot plate from the upper surface of the foot plate to the lower surface of the foot plate, and at least one recess formed in the lower surface of the foot plate about the at least one aperture of the foot plate;
a composite shank plate having a toe portion, an upper portion, an upper surface, a lower surface, at least one aperture extending through the shank plate from the upper surface of the shank plate to the lower surface of the shank plate, and at least one recess formed in the upper surface of the shank plate about the at least one aperture of the shank plate;
a composite attachment structure comprising a plurality of fibers within a resin material, the composite attachment structure attaching the shank plate to the foot plate, and comprising a portion configured to extend through the apertures of the foot plate and the shank plate, the attachment structure further comprising a first end portion and a second end portion extending from the apertures of the foot plate and the shank plate, with the first end portion forming an enlarged first end portion disposed within the at least one recess of the foot plate and the second end portion forming an enlarged second end portion disposed within the at least one recess of the shank plate.

2. The composite prosthetic foot assembly of claim 1, wherein the first end portion and the second end portion of the composite attachment structure are folded over, unidirectionally or multidirectionally, to form the enlarged first end portion and the enlarged second end portion.

3. The composite prosthetic foot assembly of claim 2, wherein the enlarged second end portion is molded to at least partially fill or completely fill or overfill the at least one recess of the shank plate and the enlarged first end portion is molded to at least partially fill or completely fill or overfill the at least one recess of the foot plate.

4. The composite prosthetic foot assembly of claim 2, wherein at least the enlarged first end portion disposed in the at least one recess of the foot plate or the enlarged second end portion disposed in the at least one recess of the shank plate is capped by at least one separate layer of composite material to at least partially fill or completely fill or overfill the at least one recess of the foot plate or the shank plate.

5. The composite prosthetic foot assembly of claim 2, wherein the at least one recess of the foot plate or the at least one recess of the shank plate is rectangular in shape and extends across a majority of the width of the foot plate or the shank plate.

6. The composite prosthetic foot assembly of 1, wherein the assembly further comprises a second composite attachment structure disposed through at least the shank plate.

7. The composite prosthetic foot assembly of claim 6, wherein the second composite attachment structure is disposed through the shank plate and the foot plate.

8. The composite prosthetic foot assembly of claim 6, wherein the second composite attachment structure is disposed through the shank plate and an adapter.

9. The composite prosthetic foot assembly of claim 8, wherein the assembly further comprises a composite backing plate disposed along the lower surface of the upper portion of the shank plate, and wherein the second composite attachment structure is configured to extend through the backing plate, the shank plate and the adapter.

10. The composite prosthetic foot assembly of claim 8, wherein the second composite attachment structure is a U-shaped structure that is configured to extend through the shank plate and the adapter.

11. The composite prosthetic foot assembly of claim 8, wherein the second composite attachment structure is a U-shaped structure that is configured to extend through the shank plate and around at least the exterior of a central body of the adapter.

12. The composite prosthetic foot assembly of claim 1, wherein the at least one recess of the foot plate, or the at least one aperture of the foot plate, or the at least one recess of the shank plate, or the at least one aperture of the shank plate include a textured inner surface.

13. The composite prosthetic foot assembly of claim 1, wherein the at least one recess and at least one aperture of the foot plate and the at least one recess and at least one aperture of the shank plate extend from the upper surface of the shank plate to the lower surface of the foot plate and form an inner opening through the foot plate and the shank plate.

14. The composite prosthetic foot assembly of claim 13, wherein the inner opening has a centrally narrowed or narrowing cross-sectional shape selected from bilaterally stepped, tapered, helical, curved, and beveled cross-sectional shapes and combinations thereof.

15. The composite prosthetic foot assembly of claim 13, wherein the composite attachment structure is molded to at least partially fill or completely fill or overfill the inner opening.

16. The composite prosthetic foot assembly of claim 1, wherein the at least one aperture of the foot plate is a plurality of apertures, and the at least one aperture of the shank plate is a plurality of apertures, and the composite attachment structure is woven through the plurality of apertures of the foot plate and the plurality of apertures of the shank plate.

17. A composite prosthetic foot assembly, comprising:
a composite heel plate having a heel portion, an upper portion, an upper surface, a lower surface, first and second apertures extending through the heel plate from the upper surface of the heel plate to the lower surface of the heel plate, and at least one recess formed in the lower surface of the heel plate about the first and second apertures;
a composite shank plate having a toe portion, an upper portion, an upper surface, a lower surface, and third and fourth apertures extending through the shank plate from the upper surface of the shank plate to the lower surface of the shank plate;
a composite attachment structure comprising a plurality of fibers within a resin material, the composite attachment structure attaching the shank plate to the heel plate, and configured in a U-shape having a first end portion and a second end portion extending transversely through at least the heel plate and the shank plate, and a connecting portion crossing the upper surface of the shank plate, with the first end portion forming an enlarged first end portion disposed within the at least one recess and the second end portion forming an enlarged second end portion disposed within the at least one recess.

18. The composite prosthetic foot assembly of claim 17, wherein the first end portion and the second end portion of the composite attachment structure are folded over, unidirectionally or multidirectionally, to form the enlarged first end portion and the enlarged second end portion.

19. The composite prosthetic foot assembly of claim 17, wherein the first enlarged end portion of the composite attachment structure disposed in the at least one recess and the second enlarged end portion of the composite attachment disposed in the at least one recess are capped by at least one separate layer of composite material to at least partially fill or completely fill or overfill the at least one recess.

20. A composite prosthetic foot assembly, comprising:
a composite foot plate having a toe portion, a heel portion, an upper surface, a lower surface, a plurality of apertures extending through the foot plate from the upper surface to the lower surface, and at least one recess formed in the lower surface of the foot plate about the plurality of apertures of the foot plate;
a composite shank plate having a toe portion, an upper portion, an upper surface, a lower surface, a plurality of apertures extending through the shank plate from the upper surface of the shank plate to the lower surface of the shank plate, and at least one recess formed in the upper surface of the shank plate about the plurality of apertures of the shank plate;
at least one composite attachment structure comprising a plurality of fibers within a resin material, the at least one composite attachment structure attaching the shank plate to the foot plate, and comprising a portion configured to extend through at least one of the plurality of apertures of the foot plate and at least one of the plurality of apertures of the shank plate, the at least one attachment structure further comprising a first end portion extending from the at least one aperture of the foot plate and a second end portion extending from the at least one aperture of the shank plate, with the first end portion forming an enlarged first end portion disposed within the at least one recess of the foot plate and the second end portion forming an enlarged second end portion disposed within the at least one recess of the shank plate.
